(19) United States
(12) Patent Application Publication
(10) Pub. No.: US 2021/0080454 A1
(43) Pub. Date: Mar. 18, 2021

(54) QUANTUM DOT BEAD HAVING MULTIFUNCTIONAL LIGAND, AND TARGET ANTIGEN DETECTION METHOD AND BIO-DIAGNOSTIC APPARATUS USING SAME

(71) Applicant: ZEUS CO., LTD., Hwaseong-si, Gyeonggi-do (KR)

(72) Inventors: Heung Su JUNG, Yongin-si, Gyeonggi-do (KR); Sung Young SHIN, Osan-si, Gyeonggi-do (KR); Hyun Soo KIM, Yongin-si, Gyeonggi-do (KR); Sang Hyun PARK, Osan-si, Gyeonggi-do (KR); Ji Young LEE, Hwaseong-si, Gyeonggi-do (KR)

(73) Assignee: ZEUS CO., LTD., Hwaseong-si, Gyeonggi-do (KR)

(21) Appl. No.: 17/044,059

(22) PCT Filed: Apr. 19, 2019
(86) PCT No.: PCT/KR2019/004774

§ 371 Date: Sep. 30, 2020

(30) Foreign Application Priority Data
May 30, 2018 (KR) 10-2018-0061879

(51) Int. Cl. G01N 33/543; B01J 20/02; B01J 20/10; B01J 20/28; B01J 20/289; B01J 20/30; B01J 20/32; G01N 33/558; G01N 21/64

(52) U.S. Cl. CPC G01N 33/543; G01N 2021/6441; B01J 20/0266; B01J 20/0244; B01J 20/103; B01J 20/28007; B01J 20/289; B01J 20/3085; B01J 20/3204; B01J 20/3219; B01J 20/3274; B01J 20/3293; G01N 33/558; G01N 21/6428; B01J 2220/54; B01J 20/0262

(57) ABSTRACT

In one aspect, the present disclosure relates to a quantum dot bead comprising a multifunctional ligand having a first binding material and a second antibody, and an immunochromatographic detection method for a target antigen in a biological sample, comprising forming multiple bonds with a quantum dot having a second binding material. In addition, the present disclosure has the effect of remarkably amplifying the detection intensity and significantly improving the detection sensitivity without a separate washing step, and thus enables the detection and diagnosis of physiological materials in a biological sample even in an actual product, and may be used to provide a product with excellent competitiveness in price.

### CROSS-REFERENCE TO RELATED APPLICATIONS

This application is based upon and claims the benefit of priority from Korean Patent Application No. 10-2018-0061879, filed on May 30, 2018, the entire contents of which are incorporated herein by reference.

### TECHNICAL FIELD

The present disclosure relates to a quantum dot bead having a multifunctional ligand, and a method of detecting a target antigen and a bio-diagnostic apparatus using the same.

### BACKGROUND

In recent years, as the medical focus is changing from treatment to diagnosis and from centralized hospital diagnosis to on-site and personal diagnosis, a diagnostic apparatus capable of directly making a diagnosis in the simplest way on site and measuring various types of diseases is in need. To implement such an apparatus, the three most necessary factors may include high sensitivity, suitable price of the diagnostic apparatus and whether multiple diagnosis is possible, and to meet these factors, various diagnostic platforms have been studied.

Currently, the most prevalent method in in vitro diagnosis is a protein-based assay, such as an immunoassay, or a nucleic acid-based molecular diagnostic technique, and these techniques are gradually encroaching on the market. That is because these techniques can amplify a target material, resulting in high sensitivity, and enable multiple diagnosis by using equipment. Nevertheless, they still have problems such as expensive equipment and reagents, a long reaction time and the need for a professional operator in order to apply them to on-site diagnosis.

Therefore, in order for them to be directly applied on site, these two techniques have limitations, where lateral flow immunoassay, which is one of the immunoassay techniques, enables on-site diagnosis in a simple way and at a low price, but has limitations in terms of the targets that can be applied due to its low sensitivity, while the molecular diagnostic method, which is the latter technique, is a method that has high sensitivity but can be used only in large laboratories due to the need for complicated equipment and professional manpower.

Thus, there is a need for a diagnostic platform that can use a platform for lateral flow immunity analysis, increase sensitivity up to the molecular diagnosis field and perform quantitative evaluations.

Fluorescent substances which are generally used in lateral flow immunoassays are gold nanoparticles that form immunocomplexes with physiological substances and develop a red color by a unique plasmon phenomenon. Due to these characteristics, these fluorescent substances have the advantage of easily detecting and identifying the presence or absence of a physiological substance from an actual product with the naked eye.

However, when gold nanoparticles are used, since the detection depends on a visual evaluation, the sensitivity is not excellent and analytical sensitivity is low, and thus the gold nanoparticles are mainly applied to physiological substances present in an excessive amount in blood. Accordingly, due to the difficulty of detecting or measuring a physiological substance present at a very low concentration in blood, there is a limit to early diagnosis of a disease. In addition, there is a problem in that a quantitative analysis of physiological substances is difficult.

Therefore, to detect a low concentration of a physiological substance, efforts to amplify the detection strength of a fluorescent substance used in lateral flow immunoassay have continued. As one of such efforts, International Patent Publication No. WO 2008-071345 discloses that gold nanoparticles are stacked using a nucleotide complementary to a colloidal gold nanoparticle, thereby amplifying their fluorescence intensities.

However, according to the above technique, gold nanoparticles having complementary nucleotides may bind to each other before conjugation with a physiological substance such as an antigen, and when the gold nanoparticles are added simultaneously, they agglomerate. Such an agglomeration phenomenon disturbs the flow of a biological sample in lateral flow immunoassay, making the detection of a target physiological material difficult. To prevent this phenomenon, before the injection of gold nanoparticles having different nucleotides, a washing step of removing conventionally existing nanoparticles is necessary. Therefore, to be applied to an actual lateral flow sensor, a washing step is required before new gold nanoparticles are added to the sensor, and thus the above technique has limitations in application to an actual sensor.

For these reasons, among fluorescent substances having a higher efficiency than gold nanoparticles and enabling multiple diagnosis, quantum dots have emerged as the strongest candidate and thus have been studied a great deal.

Recently, in the papers published by Cheng et al. (Anal Bioanal Chem, 409(1):133-141, 25 Oct. 2016), Savin et al. (Talanta. 2018 Feb. 1; 178:910-915), and Wu et al. (Analytica Chimica Acta, Volume 1008, 30 May 2018, Pages 1-7), in order to increase light efficiency, studies on increasing the efficiency by applying a quantum dot instead of a conventional gold nanoparticle or another fluorescent substance are in progress, and according to Korean Patent Application No. 10-2018-0046848 filed by ZEUS, studies on amplifying a signal by the form of a complex, rather than using a single fluorescent substance, or amplifying the detection intensity by stacking fluorescent substances to achieve higher sensitivity are in progress.

In addition, various techniques for increasing sensitivity by light amplification by a bead complex made via the stacking of fluorescent substances as suggested by Zhang et al. (Chemical Papers, 70 (8), 1031-1038, 2016) or forming a multilayer structure by 100 cycles of quantum dot reactions as suggested by Park et al. (ACSNANO, Vol 7, No 10, 9416˜9427, 2013) have been developed.

However, the bead complex has limitations in that it widens the surface area of a bead, and the increase in sensitivity through the stacking of fluorescent substances requires a separate washing procedure for each step, and thus it is difficult to apply the bead complex to on-site diagnostic equipment.

On the other hand, to exhibit sensitivity at the molecular diagnosis level by lateral flow analysis, signal amplification by stacking fluorescent substances is needed, and the implementation of this technique will be an important barometer for the success of an on-site diagnostic apparatus.

Therefore, the inventors of the present disclosure provide a detection method using a multifunctional ligand and a quantum dot bead as a technique of stably and very remarkably amplifying the detection fluorescence intensity through lateral flow immunoassay without a separate washing procedure using a method of stacking a quantum dot and a quantum dot bead, which may increase the fluorescence intensity.

### REFERENCE DOCUMENTS

1. US 2010-0068727 A1
2. WO 2008-071345 A1
3. Cheng et al., Anal Bioanal Chem, 409(1):133-141, 25 Oct. 2016
4. Savin et al., Talanta. 2018 Feb. 1; 178:910-915
5. Wu et al., Analytica Chimica Acta, Volume 1008, 30 May 2018, Pages 1-7
6. Zhang et al., Chemical Papers, 70 (8), 1031-1038, 2016
7. Park et al., ACSNANO, Vol 7, No 10, 9416˜9427, 2013

### SUMMARY

Various embodiments of the present disclosure provide a detection material to which a light amplification system is applied, which may exhibit a 100 or more stacking cycle effect without a separate washing step by simultaneously forming multiple binding of a quantum dot making a complementary bond with a multifunctional ligand of a quantum dot bead, which is a parent structure, not by sequential stacking, in order to amplify a fluorescent signal, thereby applying simple immunochromatography, providing a low-cost diagnostic platform and exhibiting sensitivity at the molecular diagnosis level, and a diagnostic method or lateral flow immunosensor using the same.

An immunochromatographic detection method for a target antigen in a biological sample according to one aspect of the present disclosure may include forming multiple bonds between a quantum dot bead including a multifunctional ligand having a large amount of first binding materials, and a second antibody, and a quantum dot having a second binding material.

According to one aspect of the present disclosure, the immunochromatographic detection method may be used in a method of diagnosing a target antigen-related disease, disorder or condition, a lateral flow immunosensor for detecting a physiological substance, and a bio-diagnostic kit.

According to the present disclosure in some embodiments, an immunochromatographic detection method can very significantly amplify detection intensity and significantly improve the detection sensitivity by a simple method without an antigen loss using a quantum dot bead having a multifunctional ligand and a quantum dot which can bind to the ligand.

The immunochromatographic detection method according to one aspect of the present disclosure can also exhibit an effect of significantly amplifying detection intensity without a continuous input of fluorescent substances for signal amplification and a separate washing step, thereby rapidly and simply detecting and identifying a physiological substance in a biological sample during actual commercialization, which is advantageous in terms of competitiveness in price.

### BRIEF DESCRIPTION OF THE DRAWINGS

FIGS. 1A and 1B are schematic diagrams showing the state in which the detection intensity is amplified by binding a quantum dot bead having a multifunctional ligand with a quantum dot that can bind to the ligand in an immunochromatographic detection method according to one aspect of the present disclosure. FIG. 1A is a schematic diagram showing a case in which an antigen-specific secondary antibody is present on the surface of a quantum dot bead, and FIG. 1B is a schematic diagram showing a case in which an antibody binds to the end of a multifunctional ligand present in a quantum dot bead.

FIG. 2 is a graph showing the zeta potentials of a quantum dot and a quantum dot bead, which are used in an immunochromatographic detection method according to one aspect of the present disclosure.

FIG. 3 is a graph showing the quantum efficiency of a quantum dot and a quantum dot bead, which may be used in an immunochromatographic detection method according to one aspect of the present disclosure.

FIGS. 4A and 4B show a transmission electron micrograph (FIG. 4A) of quantum dots and scanning electron micrographs (FIG. 4B) of quantum dot beads, which are used in an immunochromatographic detection method according to one aspect of the present disclosure.

FIG. 5 is a graph showing a particle size analysis result for quantum dot beads used in an immunochromatographic detection method according to one aspect of the present disclosure.

FIG. 6 is a graph showing fluorescence intensities when quantum dots and quantum dot beads are individually used as a comparative example, and when a quantum dot bead multi-complex is used, instead of an example of the present disclosure.

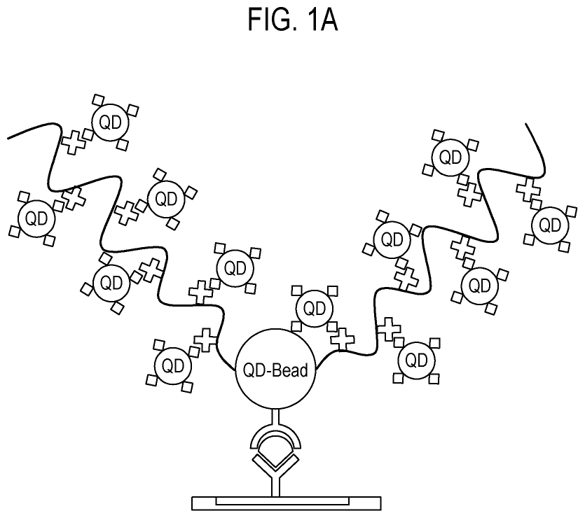

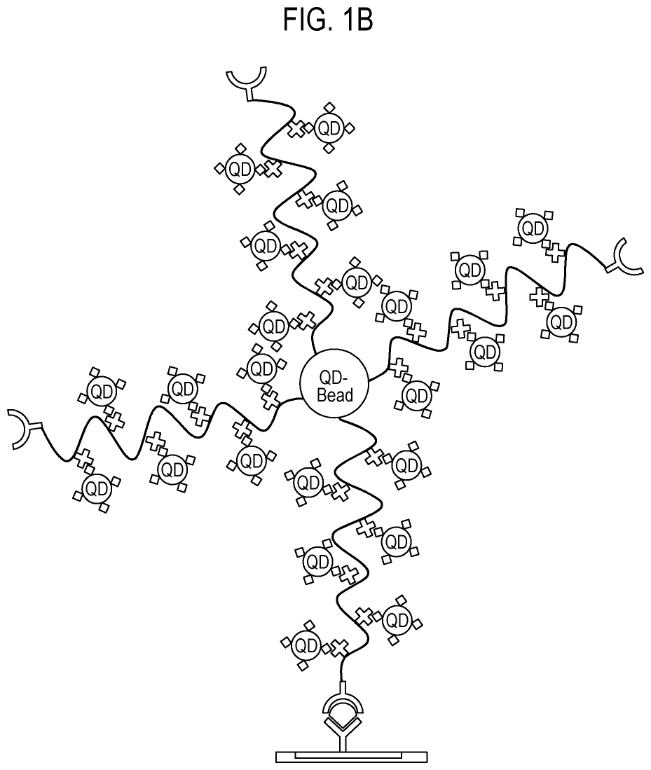

### DETAILED DESCRIPTION

In one aspect of the present disclosure, a “quantum dot” refers to a semiconductor nanoparticle, and has the characteristic of emitting different colors of light according to the size of a particle due to a quantum confinement effect. The quantum dot is approximately 20-fold brighter than a fluorescent dye such as a representative fluorescent substance, fluorescent rhodamine, and is approximately 100-fold more stable against photo-bleaching and has an approximately three-fold narrower spectral line width.

In one aspect of the present disclosure, a “quantum dot bead” is a particle including a large number of quantum dots, and is a broad concept that refers to all of particles exhibiting the characteristic of being at least 100-fold brighter than a quantum dot and prepared to include multiple quantum dots regardless of the type of core constituting the quantum dot bead.

In one aspect of the present disclosure, a “ligand” may refer to a material having a chain structure with a functional group or binding site capable of binding to a first binding material, and may also refer to a multifunctional ligand. The ligand is used to amplify fluorescence detection intensity using the first binding material. Therefore, the type of material constituting the ligand is not particularly limited, and any ligand may be used in the method of the present disclosure as long as it has a first binding material or a functional group or binding site capable of binding to an antibody. The ligand may include a first area which is the part binding to a quantum dot bead or a second antibody, a second area which forms the backbone of the ligand, and a third area which is the part binding to the first binding material. The ligand may form a covalent bond with the first binding material by a functional group or binding site, and may have one or more first binding materials. Here, the functional group may be, but is not limited to, a hydroxyl group, an amine group, a thiol group, a carbonyl group, or a carboxyl group, and any material capable of achieving conjugation with the first binding material may be used. As the first binding material on the ligand and a second binding material on a quantum dot react with or bind to each other, detection intensity may be significantly amplified. As the number of the first binding materials on the ligand increases, more quantum dots may bind to the ligand, and thus the detection intensity may be further amplified.

In one aspect of the present disclosure, a “polymer” may refer to a compound produced by polymerization of a monomer, which is a repeat unit, and means a concept within a range generally understood by those of ordinary skill in the art.

In one aspect of the present disclosure, a “nucleotide chain” may refer to a long polymer chain consisting of nucleotides, and means a concept within a range generally understood by those of ordinary skill in the art. Bases present in a nucleotide may include adenine, guanine, thymine, cytosine, uracil, and variants thereof, but the present invention is not limited thereto.

In one aspect of the present disclosure, a “peptide chain” may refer to a long polymer chain consisting of amino acids, and means a concept within a range generally understood by those of ordinary skill in the art.

In one aspect of the present disclosure, a “first binding material” and a “second binding material” may have the characteristics of binding to each other. These materials may naturally bind to each other at room temperature. For example, these materials may refer to materials that specifically bind to each other, such as an antigen and an antibody, nucleotide chains complementary to each other, an aptamer and a target material thereof, and avidin or streptavidin and biotin; or a peptide pair that can bind to each other by a hydrogen bond, a disulfide bond or a Van-der-Waals force, but the present disclosure is not limited thereto.

In one aspect of the present disclosure, the “forming multiple bonds” may refer to binding to have multiple quantum dots on one ligand.

In one aspect of the present disclosure, an “antigen” or “target antigen” is a physiological substance present in a biological sample, and a broad concept that includes all materials to be detected in connection with various diseases or the physical conditions of subjects. For example, in one aspect of the present disclosure, an antigen is a substance causing an immune response in a commonly referred biological sample, and includes all of microorganisms, viruses, etc.

In one aspect of the present disclosure, a “biological sample” is a concept encompassing all samples having a physiological environment in which an antigen can be present, for example, urine, blood, serum, plasma, and saliva, etc.

In one aspect of the present disclosure, an “antibody” is a broad concept that includes molecules inducing an immune response specifically against an antigen and binding to it so as to detect and identify the antigen. In addition, a “first antibody” and a “second antibody” recognize different epitopes of the same antigen, and is a broad concept that encompasses molecules present in pairs for antigen detection. For example, the second antibody may be fixed to a membrane of a diagnosis device to capture an antigen present in a biological sample, and the second antibody may have a detectable marker, rebind to the antigen captured by the second antibody to detect and identify the presence of the antigen in the biological sample.

In one aspect of the present disclosure, a “diameter” may refer to the length of the longest line segment passing through the center of a linker, a quantum dot or a quantum dot bead, and the average diameter may refer to the average of 10 line segments crossing the center, and in the case of the quantum dot, the diameter may refer to the size of a core-stable layer-shell layer or the size of a core-stable layer-shell-water soluble ligand layer.

Hereinafter, the present disclosure will be described in detail.

In one aspect of the present disclosure, an immunochromatographic detection method for a target antigen in a biological sample, which includes forming multiple bonds between a quantum dot bead and quantum dots, may be provided.

In one aspect of the present disclosure, the quantum dot bead may include a multifunctional ligand having a first binding material, and a second antibody.

In one aspect of the present disclosure, the quantum dot may have a second binding material.

In one aspect of the present disclosure, the first binding material and the second binding material may react with and bind to each other. In one aspect of the present disclosure, the first antibody and the second antibody may be specific for a target antigen, and these antibodies may be specific for different sites, that is, different epitopes, of a target antigen.

In one aspect of the present disclosure, the first binding material and the second binding material may be present in the ligand and the quantum dot, respectively, to allow various quantum dots to bind to the ligand. In the present disclosure, a fluorescence detection signal of the antigen is significantly amplified by binding various quantum dots to the ligand of the quantum dot bead.

In one aspect of the present disclosure, the first antibody may be attached or immobilized to a membrane, and may react with and capture the antigen present in the biological sample. The second antibody may be used to detect the antigen captured by the first antibody, and may refer to an antibody specific for a site different from the antigen-binding site of the first antibody. The second antibody may bind to the quantum dot bead, and has a quantum dot bead as a detection marker, such that the quantum dot bead may detect the captured antigen.

In one aspect of the present disclosure, the detection method may include: (a) binding a target antigen in a biological sample with a quantum dot bead; and (b) forming multiple bonds between a quantum dot bead and a quantum dot by binding a first binding material and a second binding material. In one aspect of the present disclosure, the detection method may further include, step (c) measuring fluorescence by irradiation after Step (b).

In one aspect of the present disclosure, the ligand may include a first area which is a part binding to the quantum dot bead or the second antibody, a second area which forms the backbone of the ligand, and a third area which is a part binding to the first binding material.

In one aspect of the present disclosure, the first binding material and the ligand may be covalently bound together. In one aspect of the present disclosure, the covalent bond between the first binding material and the ligand may be one or more selected from the group consisting of an ester bond, an epoxy bond, an ether bond, an imide bond, an imine bond, and an amide bond.

In one aspect of the present disclosure, the ligand may be one or more selected from the group consisting of a polymer; a nucleotide chain; and a peptide chain.

In one aspect of the present disclosure, the ligand may have one or more substituents selected from the group consisting of a hydroxyl group, an amine group, a thiol group, a carbonyl group, a carboxyl group, an epoxy group, an ethylene group, an acetylene group, an amide group, a phosphonate group, a phosphate group, a sulfonate group, a sulfate group, a nitrate group, and an ammonium group.

In one aspect of the present disclosure, the first area of the ligand may include one or more substituents selected from the group consisting of a hydroxyl group, an amine group, a thiol group, a carboxyl group, an amide group, a phosphonate group, a phosphate group, a sulfonate group, and a sulfate group.

In one aspect of the present disclosure, the third area of the ligand may include a substituent selected from the group consisting of a hydroxyl group, an amine group, a thiol group, a carboxyl group, a sulfonate group, a nitrate group, a phosphonate group, and an ammonium group.

In one aspect of the present disclosure, the polymer may be one or more selected from the group consisting of polyethylenimine, polyethylene glycol, polyacrylamide, polyphosphazene, polylactide, polylactide-co-glycolide, polycaprolactone, polyanhydride, polymalic acid and derivatives thereof, polyalkylcyanoacrylate, polyhydrooxybutylate, polycarbonate, polyorthoester, poly-L-lysine, polyglycolide, polymethyl methacrylate, polyvinylpyrrolidone, poly(vinylbenzyl trialkyl ammonium), poly(4-vinyl-N-alkyl-pyridinium), poly(acryloyl-oxyalkyl-trialkyl ammonium), poly(acrylamidoalkyl-trialkyl ammonium), poly(diallyldimethyl-ammonium), poly(styrenesulfonic acid), poly(vinyl sulfonic acid), poly(itaconic acid), a maleic acid-diallylamine copolymer, and a hyperbranched polymer.

In one aspect of the present disclosure, the nucleotide chain may consist of 10 to 500 nucleotides, but the present disclosure is not limited thereto. Specifically, in one aspect of the present disclosure, the nucleotide chain may consist of as many nucleotides it takes to have a length ranging from 10 to 100 nm, for example, 10 to 1,000 nucleotides.

In one aspect of the present disclosure, the peptide chain may consist of 10 to 500 amino acids. Specifically, in one aspect of the present disclosure, the peptide chain may consist of as many nucleotides it takes to have a length ranging from 10 to 100 nm, for example, 10 to 1,000 amino acids.

In one aspect of the present disclosure, the ligand may have a molecular weight of 100 MW (g/mol) to 1,000,000 MW. Here, the molecular weight of the ligand may correspond to the range of all integers existing in the above range. In one aspect of the present disclosure, the length of the ligand may be 2 to 10-fold the average diameter of the quantum dot bead. Specifically, in one aspect of the present disclosure, the molecular weight (MW) of the ligand may be 100 MW or more, 500 MW or more, 1,000 MW or more, 5,000 MW or more, 10,000 MW or more, 30,000 MW or more, 50,000 MW or more, 70,000 MW or more, 100,000 MW or more, 300,000 MW or more, 500,000 MW or more, or 700,000 MW or more, or 1,000,000 MW or less, 800,000 MW or less, 600,000 MW or less, 400,000 MW or less, 200,000 MW or less, 100,000 MW or less, 80,000 MW or less, 60,000 MW or less, 40,000 MW or less, 20,000 MW or less, 10,000 MW or less, 8,000 MW or less, 4,000 MW or less, 2,000 MW or less, 800 MW or less, 400 MW or less, or 200 MW or less. When the length of the ligand is more than 1 μm, in the lateral flow immunosensor, it may be a problem since it is difficult for the ligand to pass through a membrane of the immunosensor.

In one aspect of the present disclosure, the first binding material and the second binding material may be one or more selected from the group consisting of a pair of an antigen, which is not a target antigen, and an antibody, a pair of nucleotide chains, which are complementary to each other, a pair of an aptamer and a target material, a pair of peptides binding to each other, and a pair of avidin or streptavidin and biotin. In one aspect of the present disclosure, the first binding material and the second binding material may be a pair of avidin or streptavidin and biotin. In one aspect of the present disclosure, the first binding material may be biotin, and the second binding material may be avidin or streptavidin.

In one aspect of the present disclosure, the peptide pair may bind together by a hydrogen bond, a disulfide bond or a Van der Waals force.

In one aspect of the present disclosure, the second antibody may be present on the surface of a quantum dot bead or bind to a ligand at the end of the ligand.

In one aspect of the present disclosure, the quantum dot may have a core-stable layer-shell-water soluble ligand layer structure.

In one aspect of the present disclosure, the core may include one or more of cadmium (Cd) and selenium (Se); the stable layer may include one or more of cadmium (Cd), selenium (Se), zinc (Zn) and sulfur (S); and the shell may include one or more of cadmium (Cd), selenium (Se), zinc (Zn) and sulfur (S).

In one aspect of the present disclosure, the quantum dot may include one or more of Group 12 to 16 element-based compounds, Group 13 to 15-element-based compounds and Group 14 to 16 element-based compounds.

In one aspect of the present disclosure, the Group 12 to 16 element-based compounds include one or more of cadmium sulfide (CdS), cadmium selenide (CdSe), cadmium telluride (CdTe), zinc sulfide (ZnS), zinc selenide (ZnSe), zinc telluride (ZnTe), mercury sulfide (HgS), mercury selenide (HgSe), mercury telluride (HgTe), zinc oxide (ZnO), cadmium oxide (CdO), mercury oxide (HgO), cadmium selenium sulfide (CdSeS), cadmium selenium telluride (CdSeTe), cadmium sulfide telluride (CdSTe), cadmium zinc sulfide (CdZnS), cadmium zinc selenide (CdZnSe), cadmium sulfide selenide (CdSSe), cadmium zinc telluride (CdZnTe), cadmium mercury sulfide(CdHgS), cadmium mercury selenide (CdHgSe), cadmium mercury telluride (CdHgTe), zinc selenium sulfide (ZnSeS), zinc selenium telluride (ZnSeTe), zinc sulfide telluride (ZnSTe), mercury selenium sulfide (HgSeS), mercury selenium telluride (HgSeTe), mercury sulfide telluride (HgSTe), mercury zinc sulfide (HgZnS), mercury zinc selenide (HgZnSe), cadmium zinc oxide (CdZnO), cadmium mercury oxide (CdHgO), zinc mercury oxide (ZnHgO), zinc selenium oxide (ZnSeO), zinc tellurium oxide (ZnTeO), zinc sulfide oxide (ZnSO), cadmium selenium oxide (CdSeO), cadmium tellurium oxide (CdTeO), cadmium sulfide oxide (CdSO), mercury selenium oxide (HgSeO), mercury tellurium oxide (HgTeO), mercury sulfide oxide (HgSO), cadmium zinc selenium sulfide (CdZnSeS), cadmium zinc selenium telluride (CdZnSeTe), cadmium zinc sulfide telluride (CdZnSTe), cadmium mercury selenium sulfide (CdHgSeS), cadmium mercury selenium telluride (CdHgSeTe), cadmium mercury sulfide telluride (CdHgSTe), mercury zinc selenium sulfide (HgZnSeS), mercury zinc selenium telluride (HgZnSeTe), mercury zinc sulfide telluride (HgZnSTe), cadmium zinc selenium oxide (CdZnSeO), cadmium zinc tellurium oxide (CdZnTeO), cadmium zinc sulfide oxide (CdZnSO), cadmium mercury selenium oxide (CdHgSeO), cadmium mercury tellurium oxide (CdHgTeO), cadmium mercury sulfide oxide (CdHgSO), zinc mercury selenium oxide (ZnHgSeO), zinc mercury tellurium oxide (ZnHgTeO) and zinc mercury sulfide oxide (ZnHgSO), but the present disclosure is not limited thereto.

In one aspect of the present disclosure, the Group 13 to 15-element-based compounds may include one or more of gallium phosphide (GaP), gallium arsenide (GaAs), gallium antimonide (GaSb), gallium nitride (GaN), aluminum phosphide (AlP), aluminum arsenide (AlAs), aluminum antimonide (AlSb), aluminum nitride (AlN), indium phosphide (InP), indium arsenide (InAs), indium antimonide (InSb), indium nitride (InN), gallium phosphide arsenide (GaPAs), gallium phosphide antimonide (GaPSb), gallium phosphide nitride (GaPN), gallium arsenide nitride (GaAsN), gallium antimonide nitride (GaSbN), aluminum phosphide arsenide (AlPAs), aluminum phosphide antimonide (AlPSb), aluminum phosphide nitride (AlPN), aluminum arsenide nitride (AlAsN), aluminum antimonide nitride (AlSbN), indium phosphide arsenide (InPAs), indium phosphide antimonide (InPSb), indium phosphide nitride (InPN), indium arsenide nitride (InAsN), indium antimonide nitride (InSbN), aluminum gallium phosphide (AlGaP), aluminum gallium arsenide (AlGaAs), aluminum gallium antimonide (AlGaSb), aluminum gallium nitride (AlGaN), aluminum arsenide nitride (AlAsN), aluminum antimonide nitride (AlSbN), indium gallium phosphide (InGaP), indium gallium arsenide (InGaAs), indium gallium antimonide (InGaSb), indium gallium nitride (InGaN), indium arsenide nitride (InAsN), indium antimonide nitride (InSbN), aluminum indium phosphide (AlInP), aluminum indium arsenide (AlInAs), aluminum indium antimonide (AlInSb), aluminum indium nitride (AlInN), aluminum arsenide nitride (AlAsN), aluminum antimonide nitride (AlSbN), aluminum phosphide nitride (AlPN), gallium aluminum phosphide arsenide (GaAlPAs), gallium aluminum phosphide antimonide (GaAlPSb), gallium indium phosphide arsenide (GaInPAs), gallium indium aluminum arsenide (GaInAlAs), gallium aluminum phosphide nitride (GaAlPN), gallium aluminum arsenide nitride (GaAlAsN), gallium aluminum antimonide nitride (GaAlSbN), gallium indium phosphide nitride (GaInPN), gallium indium arsenide nitride (GaInAsN), gallium indium aluminum nitride (GaInAlN), gallium antimonide phosphide nitride (GaSbPN), gallium arsenide phosphide nitride (GaAsPN), gallium arsenide antimonide nitride (GaAsSbN), gallium indium phosphide antimonide (GaInPSb), gallium indium phosphide nitride (GaInPN), gallium indium antimonide nitride (GaInSbN), gallium phosphide antimonide nitride (GaPSbN), indium aluminum phosphide arsenide (InAlPAs), indium aluminum phosphide nitride (InAlPN), indium phosphide arsenide nitride (InPAsN), indium aluminum antimonide nitride (InAlSbN), indium phosphide antimonide nitride (InPSbN), indium arsenide antimonide nitride (InAsSbN) and indium aluminum phosphide antimonide (InAlPSb), but the present disclosure is not limited thereto.

In one aspect of the present disclosure, the Group 14 to 16 element-based compounds may include one or more of tin oxide (SnO), tin sulfide (SnS), tin selenide (SnSe), tin telluride (SnTe), lead sulfide (PbS), lead selenide (PbSe), lead telluride (PbTe), germanium oxide (GeO), germanium sulfide (GeS), germanium selenide (GeSe), germanium telluride (GeTe), tin selenium sulfide (SnSeS), tin selenium telluride (SnSeTe), tin sulfide telluride (SnSTe), lead selenium sulfide (PbSeS), lead selenium telluride (PbSeTe), lead sulfide telluride (PbSTe), tin lead sulfide (SnPbS), tin lead selenide (SnPbSe), tin lead telluride (SnPbTe), tin oxide sulfide (SnOS), tin oxide selenide (SnOSe), tin oxide telluride (SnOTe), germanium oxide sulfide (GeOS), germanium oxide selenide (GeOSe), germanium oxide telluride (GeOTe), tin lead sulfide selenide (SnPbSSe), tin lead selenium telluride (SnPbSeTe) and tin lead sulfide telluride (SnPbSTe), but the present disclosure is not limited thereto. In one aspect of the present disclosure, the water soluble ligand present in the water soluble ligand layer may be one or more selected from the group consisting of silica, polyethylene glycol (PEG), polyethylenimine (PEI), mercaptopropionic acid (MPA), cysteamine, mercapto-acetic acid, mercapto-undecanol, 2-mercapto-ethanol, 1-thio-glycerol, deoxyribonucleic acid (DNA), mercapto-undecanoic acid, 1-mercapto-6-phenyl-hexane, 1,16-dimecapto-hexadecane, 18-mercapto-octadecyl amine, tri-octyl phosphine, 6-mercapto-hexane, 6-mercapto-hexanoic acid, 16-mercapto-hexadecanoic acid, 18-mercapto-octadecyl amine, 6-mercapto-hexyl amine, 8-hydroxy-octylthiol, 1-thio-glycerol, mercapto-acetic acid, mercapto-undecanoic acid, hydroxamate, hydroxamic acid derivatives, ethylene diamine, glutathione, N-acetylcysteine, thioctic acid, tiopronin, mercaptosuccinic acid, dithiothreitol, dihydrolipoic acid and bucillamine, but the present disclosure is not limited thereto. In one aspect of the present disclosure, the quantum dot may consist of CdSe and ZnS.

In one aspect of the present disclosure, the average diameter of the quantum dot may be 1 to 20 nm, and specifically, 1 to 15 nm or 1 to 10 nm. Here, the average diameter of the quantum dot may correspond to the range of all integers present in the above range. Specifically, the average diameter of the quantum dot may be 1 nm or more, 2 nm or more, 3 nm or more, 4 nm or more, 5 nm or more, 6 nm or more, 7 nm or more, 8 nm or more, 9 nm or more, 10 nm or more, 15 nm or more, or 20 nm or less, 19 nm or less, 18 nm or less, 17 nm or less, 16 nm or less, 15 nm or less, 14 nm or less, 13 nm or less, 12 nm or less, 11 nm or less, or 10 nm or less.

In one aspect of the present disclosure, the average diameter of the quantum dot bead may be 50 nm to 2 Here, the average diameter of the quantum dot bead may correspond to the range of all integers present in the above range. Specifically, the average diameter of the quantum dot bead may be 50 nm or more, 100 nm or more, 120 nm or more, 140 nm or more, 160 nm or more, 180 nm or more, 200 nm or more, 250 nm or more, 300 nm or more, 400 nm or more, 450 nm or more, 500 nm or more, 700 nm or more, 900 nm or more or 1 μm or more, or 2 μm or less, 1.5 μm or less, 1 μm or less, 900 nm or less, 800 nm or less, 750 nm or less, 700 nm or less, 650 nm or less, 600 nm or less, 550 nm or less, 500 nm or less, 450 nm or less, 400 nm or less, 350 nm or less, or 300 nm or less. When the average diameter of the quantum dot bead is more than 1 it is inappropriate to use the quantum dot bead because the beads are difficult to move when used in a lateral flow sensor.

In one aspect of the present disclosure, the target antigen may be one or more selected from the group consisting of a C-reactive protein (CRP), influenza, malaria, hepatitis C virus (HCV), human immunodeficiency virus (HIV), hepatitis B virus (HBV), creatine kinase MB (CK-MB), troponin I, myoglobin, prostate specific antigen (PSA), alpha-fetoprotein (AFP), carcinoembryonic antigen (CEA), thyroid stimulating hormone (TSH), chorionic somatomammotropin hormone (CSH), human chorionic gonadotropin (hCG), cortisol, progesterone, and testosterone.

In one aspect of the present disclosure, the antigen-quantum dot bead complex produced in Step (a), before Step (b), may bind to a first antibody immobilized in the test area.

In one aspect of the present disclosure, the first antibody may be one or more selected from the group consisting of a monoclonal anti-CRP antibody, a monoclonal anti-influenza antibody, a monoclonal anti-malaria antibody, a monoclonal anti-HCV antibody, a monoclonal anti-HIV antibody, a monoclonal anti-HBV antibody, a monoclonal anti-CK-MB antibody, a monoclonal anti-troponin I antibody, a monoclonal anti-myoglobin antibody, a monoclonal anti-PSA antibody, a monoclonal anti-AFP antibody, a monoclonal anti-CEA antibody, a monoclonal anti-TSH antibody, a monoclonal anti-CSH antibody, a monoclonal anti-hCG antibody, a monoclonal anti-cortisol antibody, a monoclonal anti-progesterone antibody, and a monoclonal anti-testosterone antibody.

In one aspect of the present disclosure, the second antibody may be one or more selected from the group consisting of a polyclonal anti-CRP antibody, a polyclonal anti-influenza antibody, a polyclonal anti-malaria antibody, a polyclonal anti-HCV antibody, a polyclonal anti-HIV antibody, a polyclonal anti-HBV antibody, a polyclonal anti-CK-MB antibody, a polyclonal anti-troponin I antibody, a polyclonal anti-myoglobin antibody, a polyclonal anti-PSA antibody, a polyclonal anti-AFP antibody, a polyclonal anti-CEA antibody, a polyclonal anti-TSH antibody, a polyclonal anti-CSH antibody, a polyclonal anti-hCG antibody, a polyclonal anti-cortisol antibody, a polyclonal anti-progesterone antibody, and a polyclonal anti-testosterone antibody.

In one aspect of the present disclosure, the biological sample may be one or more selected from the group consisting of urine, blood, serum, plasma and saliva, but the present disclosure is not limited thereto.

In one aspect of the present disclosure, an immunochromatographic detection method for a target antigen in a biological sample, which includes: (a) injecting a biological sample into a first inlet; (b) binding a target antigen in the sample with a quantum dot bead including a multifunctional ligand having biotin and a second antibody by passing the same through a quantum dot bead pad while the injected biological sample is developed; (c) binding the antigen-quantum dot bead complex with a first antibody immobilized in a test area; (d) injecting a quantum dot having avidin into a second inlet; and (e) binding the quantum dot to the antigen-quantum dot bead complex present in the test area while the quantum dot is developed, may be provided.

In one aspect of the present disclosure, an immunochromatographic detection method for a target antigen in a biological sample, which includes: (a) injecting a biological sample into a first inlet; (b) binding a target antigen in the sample with a quantum dot bead including a multifunctional ligand having streptavidin or avidin and a second antibody by passing the same through a quantum dot bead pad while the injected biological sample is developed; (c) binding the antigen-quantum dot bead complex with a first antibody immobilized in a test area; (d) injecting a buffer solution into a second inlet or breaking a container containing a buffer solution by an external force to release the buffer solution into a quantum dot pad; and (e) moving a quantum dot having biotin, which is included in the quantum dot pad, to the test area while the buffer solution is developed, and binding the quantum dot to the biotin present in the ligand of the antigen-quantum dot bead complex present in the test area, may be provided.

In one aspect of the present disclosure, the immunochromatographic detection method may further include step (f) measuring the fluorescence of the quantum dot bead by irradiating the test area with UV light after Step (e).

In one aspect of the present disclosure, the buffer solution may be charged in the buffer solution container, which may be broken by an external force (e.g., pressure by a finger) to release the buffer solution into the quantum dot pad. Here, the external force refers to any type of force applied by the pressure of a finger or a structure or means for breaking the buffer solution container. When the buffer solution container is broken, the buffer solution may flow out from the buffer solution container and move or be developed to the quantum dot pad. Therefore, the quantum dot present in the quantum dot pad may be developed or move to the test area.

In one aspect of the present disclosure, the immunochromatographic detection method may further include washing the test area before Step (d). The washing step may be washing an unreacted material (e.g., an antigen and an antigen-quantum dot bead complex) in the test area.

In one aspect of the present disclosure, a method of diagnosing a target antigen-related disease, disorder or condition, which uses the immunochromatographic detection method according to one aspect of the present disclosure and further includes determining a patient's condition with respect to the target antigen from the measured fluorescence detection data, may be provided.

In one aspect of the present disclosure, a method of amplifying the fluorescence detection intensity or sensitivity of a bio-diagnostic apparatus using a quantum dot bead, which includes: bringing a target antigen in a biological sample into contact with a quantum dot bead, wherein the quantum dot bead includes a multifunctional ligand having a first binding material and a second antibody; bringing a quantum dot having a second binding material into contact with the antigen-quantum dot bead complex; and forming an antigen-quantum dot bead-quantum dot structure, in which numerous quantum dots are present on the ligand by forming multiple bonds with the ligand of the quantum dot bead, may be provided.

In one aspect of the present disclosure, a lateral flow immunosensor using the immunochromatographic detection method according to one aspect of the present disclosure may be provided.

In one aspect of the present disclosure, a bio-diagnostic apparatus for detecting a physiological material, which includes: a quantum dot bead pad which includes a quantum dot bead including a multifunctional ligand having a first binding material and a second antibody; a quantum dot pad which includes a quantum dot having a second binding material; a test pad which includes a test area in which a first antibody is immobilized; and an absorbent pad connected with the test pad, may be provided.

In one aspect of the present disclosure, the bio-diagnostic apparatus may be a lateral flow immunosensor.

In one aspect of the present disclosure, the absorbent pad may impart a capillary force to allow a fluid (e.g., a sample and a buffer solution) to be developed.

In one aspect of the present disclosure, the fluid may move to the absorbent pad by pressure.

In one aspect of the present disclosure, the bio-diagnostic device may further include a light irradiation unit irradiating the test area. In one aspect of the present disclosure, the light irradiation unit may emit UV light. The light irradiation unit may facilitate easy confirmation of the antigen-antibody reaction in the test, and induces the fluorescence of the quantum dot bead in the test area. Accordingly, the presence or absence of the target antigen may be measured/detected.

In one aspect of the present disclosure, the bio-diagnostic apparatus may further include a buffer solution container, which may be separately present in the diagnostic apparatus. The buffer solution container may be broken by an external force to release the buffer solution, and when the buffer solution container is broken, the buffer solution may be developed to the quantum dot pad or the buffer solution pad.

Hereinafter, the configuration and effects of the present disclosure will be described in further detail with reference to examples and experimental examples. However, these examples and comparative examples are merely provided to help in the understanding of the present disclosure, and the scope and range of the present disclosure are not limited to the following examples.

### [Preparation Example 1] Preparation of Quantum Dot Having Biotin on its Surface

(1) Preparation of Oil Soluble Quantum Dot.

In a 3-neck flask, 1.0 g of zinc acetate (Zn(Ac)₂), 0.441 g of cadmium oxide (CdO), 20 mL of oleic acid and 75 mL of octadecene (ODE) were mixed, and water was removed at 150° C. for 1 hour under a nitrogen atmosphere. Subsequently, the resulting flask was heated to 300° C., and then 1 mL of trioctylphosphine (TOP) and 0.045 g of selenium (Se) were injected and heated for 3 minutes, thereby forming the core of a quantum dot.

Afterward, 0.5 mL of dodecanethiol was added to the 3-neck flask and reacted for 10 minutes. A solution containing 1 mL of TOP and 0.025 g of sulfur (S) was then added to the reaction vessel of the 3-neck flask and reacted for 20 minutes, thereby forming a shell. Then, the resulting core and shell were purified with a mixed solution of ethanol and toluene and dissolved in an organic solvent, thereby obtaining a primary quantum dot.

0.5 g of the resulting primary quantum dots, 1 g of zinc acetate, 0.21 g of cadmium oxide, 10 mL of oleic acid and 35 mL of octadecene were put into a separate 3-neck flask, and reacted at 300° C. for 30 minutes. Subsequently, 0.5 mL of octanethiol was added and stirred for 10 minutes, a solution containing 1 mL of TOP and 0.025 g of sulfur was put into the reaction vessel of the 3-neck flask and reacted for 20 minutes. Afterward, the resulting mixture was purified with a mixed solution of ethanol and toluene and dissolved in an organic solvent, thereby obtaining a secondary quantum dot. Such a quantum dot has a core-stable layer-shell-oil soluble ligand layer structure.

(2) Preparation of Carboxyl Group-Substituted Water Soluble Quantum Dots

20 mg of the secondary quantum dots were added to a reaction vessel containing 1 mL mercaptopropionic acid (MPA) and reacted at 60° C. for 60 minutes, thereby obtaining the final quantum dots having a water soluble ligand (carboxyl group).

(3) Preparation of PEI-Substituted Water Soluble Quantum Dots

PEI was mixed with tetrahydrofuran (“THF”), thereby preparing an 80 mg/mL PEI solution.

0.25 μL of the secondary quantum dots of Preparation Example 1-(1) having a concentration of 10 mg/mL was mixed with 400 μL of THF, 500 μL of the PEI-THF solution was slowly added thereto, and then reacted at room temperature overnight. Afterward, the resulting product was purified with THF and dissolved in distilled water, thereby preparing quantum dots (PEI-quantum dot) having an amine group.

(4) Preparation of Quantum Dots Having Biotin

Biotin having a concentration 10-fold higher than that of the quantum dots (based on mole number), 1-ethyl-3-(3-dimethylaminopropyl)carbodiimide (EDC) and N-hydroxysuccinimide (NETS) were mixed, and reacted for 2 hours at room temperature. After the reaction, the resulting mixture was centrifuged, washed with tertiary distilled water three times, and then reacted with the quantum dots of Preparation Example 1-(3) for 1 hour at room temperature. After the reaction, the resulting product was centrifuged, washed with tertiary distilled water three times, treated with bovine serum albumin (BSA), followed by a reaction for 1 hour at room temperature.

After the reaction, the resulting product was centrifuged, washed with tertiary distilled water three times, and stored by dispersing in a solution containing a 1M Tris buffer (pH 8) and 0.1% BSA.

### [Preparation Example 2] Preparation of Quantum Dot Beads Having a Multifunctional Ligand Bound to Avidin

(1) Synthesis of Silica Particle Substrate and Surface Modification

A silica-based nanoparticle was synthesized by the Stober process. First, NH₄OH, EtOH and H₂O were stirred in a ratio of 3:60:1 mL in an Erlenmeyer flask, 2 mL of tetraethyl orthosilicate (TEOS) was added to the reactants, and the mixture was stirred at 50° C. and reacted for 18 hours or more. Here, the reaction time and the mixing ratio may be adjusted according to the desired size. Subsequently, a final sample was obtained by a centrifuge using ethanol. Here, approximately 200 nm of silica beads may be obtained.

Subsequently, for a reaction between the surface and the quantum dots, as a reactive functional group, 180 μL each of 3-mercaptopropyltrimethoxysilane (MPTS) and NH₄OH was added, and reacted for 12 to 24 hours. After purification by a centrifuge using ethanol, a surface-modified silica particle substrate was obtained.

(2) Binding Quantum Dots to the Substrate

A ratio of the quantum dots of Preparation Example 1-(1) and the surface-modified silica substrate was 50:100 (mg), chloroform was added at a volume two-fold higher than the above mixture and then stirred, followed by a reaction for 30 minutes. After the reaction, a quantum dot bead was obtained.

(3) Surface Modification of Quantum Dot Beads

The CdSe/ZnS quantum dot beads synthesized in Preparation Example 2-(2) and MPA (50 mg:20 μL) were mixed with chloroform and ethanol (2 mL:2 mL) and reacted by mixing for 10 hours, and a water soluble ligand, which is a carboxyl group, was attached to the outer surface of the final quantum dot bead to modify the surface, followed by purification using ethanol and a centrifuge.

(4) Preparation of Quantum Dot Beads Having an Antibody

0.1 nmol of the quantum dot beads (—COOH) synthesized in Preparation Example 2-(3), EDC and NHS were input, and reacted with a vortex for 2 hours.

After the reaction, the resulting mixture was centrifuged to spin down the quantum dot beads (—COOH), and then the quantum dot beads (—COOH) were dispersed in PBS. Subsequently, a polyclonal anti-CRP antibody (Invitrogen) was added to have a concentration (based on mole number) 10-fold higher than that of the quantum dot beads (—COOH), and reacted for 1 hour.

After the reaction, the resulting product was washed with Tween 20 phosphate buffered saline (TPBS) twice and PBS (pH 7.4) once. Subsequently, the resulting product was dispersed in 1 mL of 5% BSA, and reacted with a vortex for 1 hour. After the reaction, the resulting product was washed with TPBS twice and PBS (pH 7.4) once.

(5) Synthesis of the Ligand (Hyperbranched Polymer; HBP)

0.25 g of p-phenylenediamine (PD), 0.52 g of trimesic acid (TMA), 2 mL of pyridine (Py), and 20 mL of N-methylpyrrolidone (NMP) were put into a 3-neck flask, and mixed under a nitrogen stream. Subsequently, 4 mL of triphenylphosphine (TPP) was slowly added thereto, followed by a reaction at 80° C. for 3 hours. Subsequently, the resulting product was purified with methanol, thereby obtaining HBP.

(6) Preparation of a Multifunctional Ligand Bound to Streptavidin

EDC and NHS were added to the long ligand (—COOH) prepared in Preparation Example 2-(5), and reacted with a vortex for 2 hours. After the reaction, the resulting mixture was washed with distilled water (d.w.) three times, and then dispersed in PBS (pH 7.4). Subsequently, streptavidin was added to obtain a concentration (based on mole number) 100-fold higher than that of the ligand (—COOH), and then reacted for 1 hour. After the reaction, the resulting product was washed with distilled water (d.w.) three times, mixed with 10% ethanolamine and then reacted for 1 hour. After the reaction, the resulting product was washed with distilled water (D.W) three times, and then stored by dispersion in PBS (pH 7.4).

(7) Preparation of Quantum Dot Beads Having a Multifunctional Ligand Bound to Streptavidin and Antibody

The quantum dot beads (—COOH) having an antibody prepared in Preparation Example 2-(4) and the multifunctional ligand bound to streptavidin (—SH) prepared in Preparation Example 2-(6) were mixed, and reacted with a vortex for 1 hour. After the reaction, the quantum dot beads were centrifuged, washed with distilled water three times, and then stored by dispersion in PBS (pH 7.4).

### [Experimental Example 1] Confirmation of the Characteristics of Quantum Dots and Quantum Dot Beads

(1) The Zeta Potential of Quantum Dots and Quantum Efficiency of Quantum Dots and Quantum Dot Beads

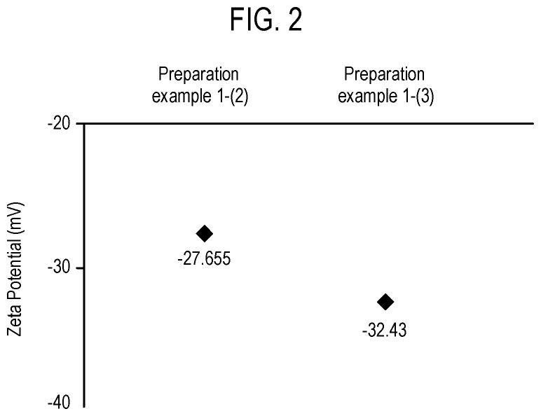

The zeta potentials of the quantum dots of Preparation Examples 1-(2) and 1-(3) were measured using ELS-100ZS (Otsuka Corp.), and the result is shown in FIG. 2.

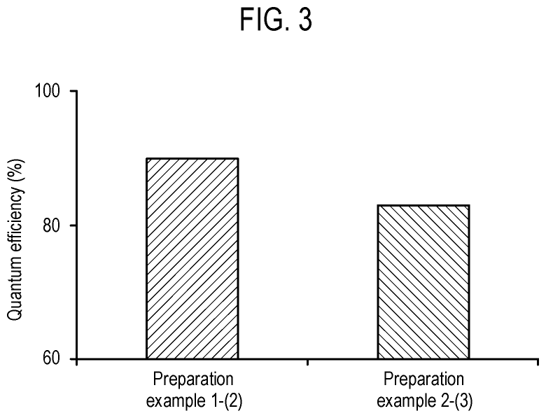

The quantum efficiency of the quantum dots of Preparation Example 1-(2) and the quantum dot beads of Preparation Example 2-(3) was measured using QE 2000 (Otsuka Corp.), and the result is shown in FIG. 3. According to these results, the quantum dots and the quantum dot beads, which were prepared in Preparation Examples 1-(2) and 2-(3) according to one aspect of the present disclosure, exhibited a quantum efficiency of 92±3% and 83±3%, respectively, and as both are over 80%, exhibited excellent effects.

(2) Confirmation of Sizes and Shapes of Quantum Dots and Quantum Dot Beads

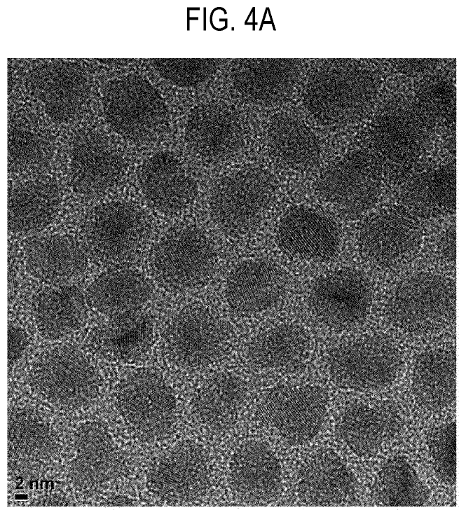

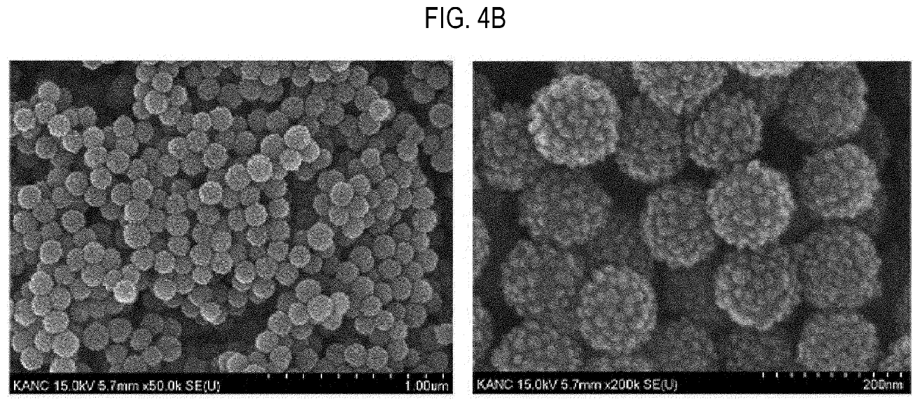

To determine the sizes and shapes of the quantum dots of Preparation Example 1-(1) and the quantum dot beads of Preparation Example 2-(2), JEM-2100F (JEOL Ltd.) and FE-SEM (Hitachi Corp.) were used, and a transmission electron micrograph of the quantum dots is shown in FIG. 4A, and a scanning electron micrograph of the quantum dot beads is shown in FIG. 4B. According to these results, it can be confirmed that both the quantum dots and the quantum dot beads according to one aspect of the present disclosure have a spherical shape with a uniform size.

(3) Particle Size Analysis of Quantum Dot Beads

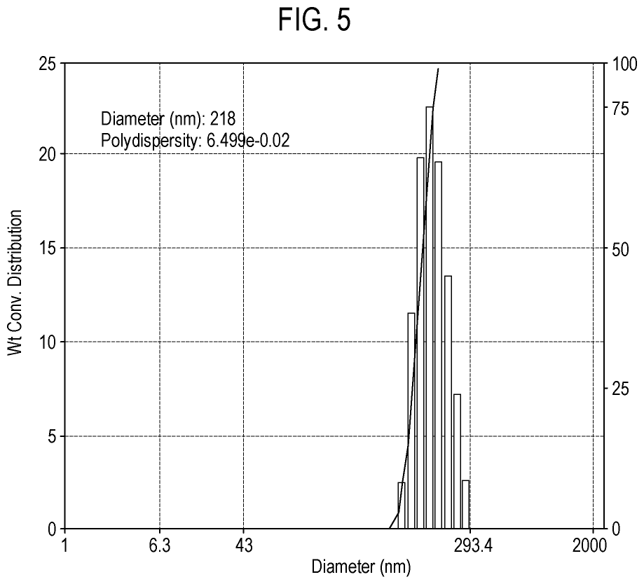

The particle size analysis of the quantum dot beads of Preparation Example 2-(2) was performed using ELS100 (Otsuka Corp.), and the result is shown in FIG. 5. From the result, it can be confirmed that the quantum dot bead that can be used herein exhibits high polydispersity. When nano-fluorescent substances agglomerate, the efficiency may deteriorate and the problem of non-specific noise may occur. Therefore, whether or not the original size is maintained is an important factor in the use of the bead as a fluorescent substance. Since the quantum dot beads that can be used herein exhibit high polydispersity, the above-mentioned problem may not occur.

### [Experimental Example 2] Experiment to Confirm the Fluorescent Reactivity in a Lateral Flow Immunosensor

### Comparative Examples 1 and 2

3 pmol (1 μL) of a polyclonal anti-CRP antibody (Invitrogen Corp.) was injected into a nitrocellulose (NC) membrane test area of a biosensor and then dried. During the preparation of the quantum dots of Preparation Example 1-(4), in Comparative Example 1, a quantum dot reacting with the polyclonal anti-CRP antibody instead of biotin, and in Comparative Example 2, the quantum dot bead of Preparation Example 2-(4) binding to the polyclonal anti-CRP antibody, were injected into a conjugate pad and then dried.

A CRP antigen (0.001 ng/mL, 0.1 ng/mL or 10 ng/mL; Invitrogen Corp.) was put into a first inlet and developed for 5 minutes. After advancing, the fluorescence intensity of the biosensor was measured using a QD-J7 fluorescent analyzer.

### Example

3 pmol (1 μL) of a monoclonal anti-CRP antibody (Invitrogen Corp.) was injected into a NC membrane test area of a biosensor and then dried.

The quantum dot beads of Preparation Example 2-(7) were injected into a conjugate pad and then dried. A CRP antigen (0.001 ng/mL, 0.1 ng/mL or 10 ng/mL; Invitrogen Corp.) was put into a first inlet and developed for 5 minutes, and then the quantum dot of Preparation Example 1-(4) was put into a second inlet to develop the resulting solution for 10 minutes. After development, the fluorescence intensity of the biosensor was measured using a QD-J7 fluorescent analyzer.

<Results>

According to a detection method using both the quantum dot bead having the multifunctional ligand bound to streptavidin and an antibody and the quantum dot having biotin according to one aspect of the present disclosure, it can be confirmed that, in all antigen concentration ranges, the sensitivity or fluorescence intensity for detecting the antigen was at least 10-fold higher than when a quantum dot and a quantum dot bead were individually used.

Compared to the quantum dot of Comparative Example 1, since the quantum dot bead of Comparative Example 2 contains at least 200 to 500-fold more quantum dots, the fluorescence detection intensity or detection sensitivity should correspondingly increase, but is actually similar to that using the quantum dot of Comparative Example 1. That is because the number of second antibodies (e.g., polyclonal anti-CRP antibodies) binding to one quantum dot bead increases similarly to the increasing number of quantum dots, resulting in a reduction in the number of detected antigens and a decrease in detection intensity. On the other hand, according to the method of the present disclosure, the detection intensity may be very significantly amplified by binding the multifunctional ligand having multiple streptavidins to the quantum dot bead, and binding the quantum dots having biotin, which specifically binds to streptavidin, thereto. The method of the present disclosure may very remarkably amplify detection intensity by a simple method without a separate washing step.

### [Experimental Example 3] Simulation for the Amplification of Fluorescence Intensity in the Present Disclosure

To confirm a degree of the amplification of fluorescence intensity in the present disclosure, an additional experiment was performed. The quantum dot used in Comparative Example 1 and a quantum dot bead multi-complex which is expected to exhibit similar fluorescence intensity amplification to the above example were used.

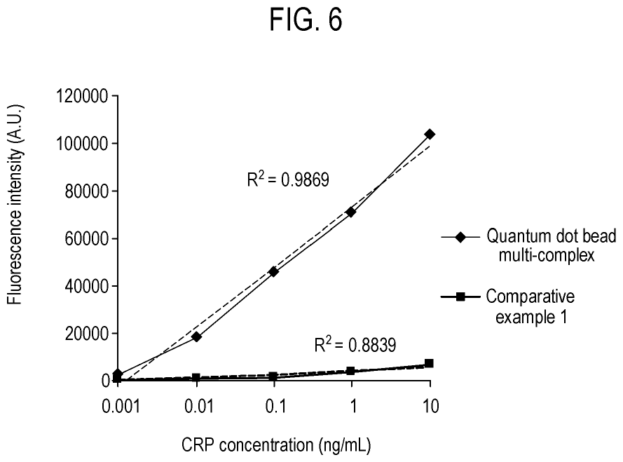

Specifically, the quantum dot bead multi-complex was prepared by binding numerous small quantum dot beads having a diameter of 100 to 300 nm to the large quantum dot bead having a diameter of 1 μm or more as the quantum dot bead prepared in Preparation Example 2-(3), and then a polyclonal anti-CRP antibody was bound to the complex. The resulting quantum dot bead multi-complex and the quantum dot of Comparative Example 1 were put into separate wells of a well plate, and then a CRP antigen (0.001 ng/mL, 0.01 ng/mL, 0.1 ng/mL, 1 ng/mL and 10 ng/mL; Invitrogen Corp.) were added to the respective wells. Subsequently, the fluorescence intensity was measured using a fluorescence spectrometer (FS-2, SCINCO, Ltd.), and the result is shown in FIG. 6.

According to FIG. 6, compared to Comparative Example 1 only using a quantum dot, it can be confirmed that the fluorescence intensity of the quantum dot bead multi-complex, which replaces the example of the present disclosure, is very remarkably amplified and very superior. Specifically, at a CRP concentration of 10 ng/mL, the quantum dot bead multi-complex exhibited an approximately 500-fold higher fluorescence intensity than that of the quantum dot of Comparative Example 1, demonstrating that when a target antigen is detected according to the present disclosure the target antigen can be detected with a 500-fold higher sensitivity, compared to Comparative Example 1.

As above, as specific parts of the specification have been described in detail, although it is clear to those skilled in the art that this specific technique is merely a preferred embodiment, the scope of the specification is not limited thereto. Thus, the substantial scope of the specification will be defined by the accompanying claims and their equivalents.

1. An immunochromatographic detection method for a target antigen in a biological sample, comprising:
forming multiple bonds between a quantum dot bead including a multifunctional ligand having a first binding material and a second antibody, and a quantum dot having a second binding material,
wherein the first binding material and the second binding material react to bind to each other, and the second antibody is specific for a target antigen.

2. The method of claim 1, comprising:
(a) binding a target antigen in a biological sample with a quantum dot bead; and
(b) forming multiple bonds between the quantum dot bead and quantum dots by bonding a first binding material and a second binding material.

3. The method of claim 2, further comprising:
after Step (b), Step (c) measuring fluorescence by UV irradiation.

4. The method of claim 1, wherein the first binding material and the multifunctional ligand are covalently bonded.

5. The method of claim 1, wherein the multifunctional ligand is a polymer; a nucleotide chain; or a peptide chain.

6. The method of claim 5, wherein the multifunctional ligand has one or more substituents selected from the group consisting of a hydroxyl group, an amine group, a thiol group, a carbonyl group, a carboxyl group, an epoxy group, an ethylene group, an acetylene group, an amide group, a phosphonate group, a phosphate group, a sulfonate group, a sulfate group, a nitrate group, and an ammonium group.

7. The method of claim 5, wherein the multifunctional ligand includes a first area bound to the quantum dot bead and a third area bound to the first binding material,
the first area includes one or more substituents selected from the group consisting of a hydroxyl group, an amine group, a thiol group, a carboxyl group, an amide group, a phosphonate group, a phosphate group, a sulfonate group, and a sulfate group, and
the third area includes a substituent selected from the group consisting of a hydroxyl group, an amine group, a thiol group, a carboxyl group, a sulfonate group, a nitrate group, a phosphonate group, and an ammonium group.

8. The method of claim 5, wherein the multifunctional ligand is a polymer, and the polymer is one or more selected from the group consisting of polyethylenimine, polyethylene glycol, polyacrylamide, polyphosphazene, polylactide, polylactide-co-glycolide, polycaprolactone, polyanhydride, polymalic acid and derivatives thereof, polyalkylcyanoacrylate, polyhydrooxybutylate, polycarbonate, polyorthoester, poly-L-lysine, polyglycolide, polymethyl methacrylate, polyvinylpyrrolidone, poly(vinylbenzyl trialkyl ammonium), poly(4-vinyl-N-alkyl-pyridinium), poly(acryloyl-oxyalkyl-trialkyl ammonium), poly(acrylamidoalkyl-trialkyl ammonium), poly(diallyldimethyl-ammonium), poly(styrenesulfonic acid), poly(vinyl sulfonic acid), poly(itaconic acid), a maleic acid-diallylamine copolymer, and a hyperbranched polymer.

9. The method of claim 5, wherein the multifunctional ligand is a nucleotide chain, which consists of 10 to 500 nucleotides.

10. The method of claim 5, wherein the multifunctional ligand is a peptide chain, which consists of 10 to 500 amino acids.

11. The method of claim 1, wherein the multifunctional ligand has a molecular weight of 100 to 1,000,000 MW (g/mol).

12. The method of claim 1, wherein the first binding material and the second binding material are one or more selected from the group consisting of a pair of an antigen which is not a target antigen, and an antibody, a pair of nucleotide chains, which are complementary to each other, a pair of an aptamer and a target material, a pair of peptides binding to each other, and a pair of avidin or streptavidin and biotin.

13. The method of claim 12, wherein the first binding material and the second binding material are a pair of avidin or streptavidin and biotin.

14. The method of claim 13, wherein the first binding material is avidin or streptavidin, and the second binding material is biotin.

15. The method of claim 12, wherein the peptide pair binds together by a hydrogen bond, a disulfide bond or a Van der Waals force.

16. The method of claim 1, wherein the second antibody is present on the surface of the quantum dot bead or at the end of the multifunctional ligand.

17. The method of claim 1, wherein the quantum dot has a core-stable layer-shell-water soluble ligand layer structure.

18. The method of claim 17, wherein the core includes one or more of cadmium (Cd) and selenium (Se),
the stable layer includes one or more of cadmium (Cd), selenium (Se), zinc (Zn) and sulfur (S), and
the shell includes one or more of cadmium (Cd), selenium (Se), zinc (Zn) and sulfur (S).

19. The method of claim 1, wherein the quantum dot includes one or more of Group 12 to 16 element-based compounds, Group 13 to 15-element-based compounds and Group 14 to 16 element-based compounds.

20. The method of claim 19, wherein the Group 12 to 16 element-based compounds include one or more of cadmium sulfide (CdS), cadmium selenide (CdSe), cadmium telluride (CdTe), zinc sulfide (ZnS), zinc selenide (ZnSe), zinc telluride (ZnTe), mercury sulfide (HgS), mercury selenide (HgSe), mercury telluride (HgTe), zinc oxide (ZnO), cadmium oxide (CdO), mercury oxide (HgO), cadmium selenium sulfide (CdSeS), cadmium selenium telluride (CdSeTe), cadmium sulfide telluride (CdSTe), cadmium zinc sulfide (CdZnS), cadmium zinc selenide (CdZnSe), cadmium sulfide selenide (CdSSe), cadmium zinc telluride (CdZnTe), cadmium mercury sulfide(CdHgS), cadmium mercury selenide (CdHgSe), cadmium mercury telluride (CdHgTe), zinc selenium sulfide (ZnSeS), zinc selenium telluride (ZnSeTe), zinc sulfide telluride (ZnSTe), mercury selenium sulfide (HgSeS), mercury selenium telluride (HgSeTe), mercury sulfide telluride (HgSTe), mercury zinc sulfide (HgZnS), mercury zinc selenide (HgZnSe), cadmium zinc oxide (CdZnO), cadmium mercury oxide (CdHgO), zinc mercury oxide (ZnHgO), zinc selenium oxide (ZnSeO), zinc tellurium oxide (ZnTeO), zinc sulfide oxide (ZnSO), cadmium selenium oxide (CdSeO), cadmium tellurium oxide (CdTeO), cadmium sulfide oxide (CdSO), mercury selenium oxide (HgSeO), mercury tellurium oxide (HgTeO), mercury sulfide oxide (HgSO), cadmium zinc selenium sulfide (CdZnSeS), cadmium zinc selenium telluride (CdZnSeTe), cadmium zinc sulfide telluride (CdZnSTe), cadmium mercury selenium sulfide (CdHgSeS), cadmium mercury selenium telluride (CdHgSeTe), cadmium mercury sulfide telluride (CdHgSTe), mercury zinc selenium sulfide (HgZnSeS), mercury zinc selenium telluride (HgZnSeTe), mercury zinc sulfide telluride (HgZnSTe), cadmium zinc selenium oxide (CdZnSeO), cadmium zinc tellurium oxide (CdZnTeO), cadmium zinc sulfide oxide (CdZnSO), cadmium mercury selenium oxide (CdHgSeO), cadmium mercury tellurium oxide (CdHgTeO), cadmium mercury sulfide oxide (CdHgSO), zinc mercury selenium oxide (ZnHgSeO), zinc mercury tellurium oxide (ZnHgTeO) and zinc mercury sulfide oxide (ZnHgSO).

21. The method of claim 19, wherein the Group 13 to 15-element-based compounds include one or more of gallium phosphide (GaP), gallium arsenide (GaAs), gallium antimonide (GaSb), gallium nitride (GaN), aluminum phosphide (AlP), aluminum arsenide (AlAs), aluminum antimonide (AlSb), aluminum nitride (AlN), indium phosphide (InP), indium arsenide (InAs), indium antimonide (InSb), indium nitride (InN), gallium phosphide arsenide (GaPAs), gallium phosphide antimonide (GaPSb), gallium phosphide nitride (GaPN), gallium arsenide nitride (GaAsN), gallium antimonide nitride (GaSbN), aluminum phosphide arsenide (AlPAs), aluminum phosphide antimonide (AlPSb), aluminum phosphide nitride (AlPN), aluminum arsenide nitride (AlAsN), aluminum antimonide nitride (AlSbN), indium phosphide arsenide (InPAs), indium phosphide antimonide (InPSb), indium phosphide nitride (InPN), indium arsenide nitride (InAsN), indium antimonide nitride (InSbN), aluminum gallium phosphide (AlGaP), aluminum gallium arsenide (AlGaAs), aluminum gallium antimonide (AlGaSb), aluminum gallium nitride (AlGaN), aluminum arsenide nitride (AlAsN), aluminum antimonide nitride (AlSbN), indium gallium phosphide (InGaP), indium gallium arsenide (InGaAs), indium gallium antimonide (InGaSb), indium gallium nitride (InGaN), indium arsenide nitride (InAsN), indium antimonide nitride (InSbN), aluminum indium phosphide (AlInP), aluminum indium arsenide (AlInAs), aluminum indium antimonide (AlInSb), aluminum indium nitride (AlInN), aluminum arsenide nitride (AlAsN), aluminum antimonide nitride (AlSbN), aluminum phosphide nitride (AlPN), gallium aluminum phosphide arsenide (GaAlPAs), gallium aluminum phosphide antimonide (GaAlPSb), gallium indium phosphide arsenide (GaInPAs), gallium indium aluminum arsenide (GaInAlAs), gallium aluminum phosphide nitride (GaAlPN), gallium aluminum arsenide nitride (GaAlAsN), gallium aluminum antimonide nitride (GaAlSbN), gallium indium phosphide nitride (GaInPN), gallium indium arsenide nitride (GaInAsN), gallium indium aluminum nitride (GaInAlN), gallium antimonide phosphide nitride (GaSbPN), gallium arsenide phosphide nitride (GaAsPN), gallium arsenide antimonide nitride (GaAsSbN), gallium indium phosphide antimonide (GaInPSb), gallium indium phosphide nitride (GaInPN), gallium indium antimonide nitride (GaInSbN), gallium phosphide antimonide nitride (GaPSbN), indium aluminum phosphide arsenide (InAlPAs), indium aluminum phosphide nitride (InAlPN), indium phosphide arsenide nitride (InPAsN), indium aluminum antimonide nitride (InAlSbN), indium phosphide antimonide nitride (InPSbN), indium arsenide antimonide nitride (InAsSbN) and indium aluminum phosphide antimonide (InAlPSb).

22. The method of claim 19, wherein the Group 14 to 16 element-based compounds include one or more of tin oxide (SnO), tin sulfide (SnS), tin selenide (SnSe), tin telluride (SnTe), lead sulfide (PbS), lead selenide (PbSe), lead telluride (PbTe), germanium oxide (GeO), germanium sulfide (GeS), germanium selenide (GeSe), germanium telluride (GeTe), tin selenium sulfide (SnSeS), tin selenium telluride (SnSeTe), tin sulfide telluride (SnSTe), lead selenium sulfide (PbSeS), lead selenium telluride (PbSeTe), lead sulfide telluride (PbSTe), tin lead sulfide (SnPbS), tin lead selenide (SnPbSe), tin lead telluride (SnPbTe), tin oxide sulfide (SnOS), tin oxide selenide (SnOSe), tin oxide telluride (SnOTe), germanium oxide sulfide (GeOS), germanium oxide selenide (GeOSe), germanium oxide telluride (GeOTe), tin lead sulfide selenide (SnPbSSe), tin lead selenium telluride (SnPbSeTe) and tin lead sulfide telluride (SnPbSTe).

23. The method of claim 20, wherein the quantum dot consists of CdSe and ZnS.

24. The method of claim 1, wherein the quantum dot bead has an average diameter of 50 nm to 2 μm.

25. (canceled)

26. The method of claim 1, wherein the quantum dot has an average diameter of 1 to 20 nm.

27. The method of claim 1, wherein the target antigen is one or more selected from the group consisting of a C-reactive protein (CRP), influenza, malaria, hepatitis C virus (HCV), human immunodeficiency virus (HIV), hepatitis B virus (HBV), creatine kinase MB (CK-MB), troponin I, myoglobin, prostate specific antigen (PSA), alpha-fetoprotein (AFP), carcinoembryonic antigen (CEA), thyroid stimulating hormone (TSH), chorionic somatomammotropin hormone (CSH), human chorionic gonadotropin (hCG), cortisol, progesterone, and testosterone.

28. The method of claim 1, wherein the second antibody is one or more selected from the group consisting of a polyclonal anti-CRP antibody, a polyclonal anti-influenza antibody, a polyclonal anti-malaria antibody, a polyclonal anti-HCV antibody, a polyclonal anti-HIV antibody, a polyclonal anti-HBV antibody, a polyclonal anti-CK-MB antibody, a polyclonal anti-troponin I antibody, a polyclonal anti-myoglobin antibody, a polyclonal anti-PSA antibody, a polyclonal anti-AFP antibody, a polyclonal anti-CEA antibody, a polyclonal anti-TSH antibody, a polyclonal anti-CSH antibody, a polyclonal anti-hCG antibody, a polyclonal anti-cortisol antibody, a polyclonal anti-progesterone antibody, and a polyclonal anti-testosterone antibody.

29. The method of claim 2, wherein the antigen-quantum dot bead complex produced in Step (a) is conjugated with a first antibody immobilized in a test area before Step (b), and the first antibody is one or more selected from the group consisting of a monoclonal anti-CRP antibody, a monoclonal anti-influenza antibody, a monoclonal anti-malaria antibody, a monoclonal anti-HCV antibody, a monoclonal anti-HIV antibody, a monoclonal anti-HBV antibody, a monoclonal anti-CK-MB antibody, a monoclonal anti-troponin I antibody, a monoclonal anti-myoglobin antibody, a monoclonal anti-PSA antibody, a monoclonal anti-AFP antibody, a monoclonal anti-CEA antibody, a monoclonal anti-TSH antibody, a monoclonal anti-CSH antibody, a monoclonal anti-hCG antibody, a monoclonal anti-cortisol antibody, a monoclonal anti-progesterone antibody, and a monoclonal anti-testosterone antibody.

30. The method of claim 1, wherein the biological sample is selected from the group consisting of urine, blood, serum, plasma and saliva.

31-34. (canceled)

35. A method of diagnosing a target antigen-related disease, disorder or condition, which uses the detection method of claim 1, and
further comprises determining a patient's condition with respect to the target antigen from the measured fluorescence detection data.

36. A lateral flow immunosensor which uses the detection method of claim 1.

37-38. (canceled)
